(19) United States
(12) Patent Application Publication
(10) Pub. No.: US 2014/0350345 A1
(43) Pub. Date: Nov. 27, 2014

(54) DEVICE TO ACCOMMODATE MULTIPLE PORTS THROUGH A SINGLE INCISION FOR LAPAROSCOPIC SURGERY

(71) Applicant: THOMAS FREDERICK HELMSWORTH, MUSCATINE, IA (US)

(72) Inventor: THOMAS FREDERICK HELMSWORTH, MUSCATINE, IA (US)

(21) Appl. No.: 13/901,395

(22) Filed: May 23, 2013

(51) Int. Cl. A61B 17/34

(52) U.S. Cl. CPC A61B 17/3423
USPC 600/204

(57) ABSTRACT

A device to accommodate multiple ports through a single incision for laparoscopic surgery for replacing the three to four incisions required by standard laparoscopic surgeries with a single incision. The device to accommodate multiple ports through a single incision for laparoscopic surgery includes an incision access member having a cylindrical body with bores for accommodating various surgical instruments inside a person's body through an incision in a body cavity wall of a person.

### CROSS-REFERENCE TO RELATED APPLICATIONS

The present application claims benefit of U.S. provisional application Ser. No. 61/513,396, filed on Jul. 29, 2011, the disclosure of which is expressly incorporated by reference herein in its entirety.

### BACKGROUND OF THE INVENTION

1. Field of the Invention

The present invention relates to surgical aids and more particularly pertains to a new device to accommodate multiple ports through a single incision for laparoscopic surgery.

The single incision laparoscopic surgery (SILS) procedure replaces the multiple incisions required by standard laparoscopic surgeries with a single incision. The current SILS procedure requires that all the tools enter the same point, making the surgery difficult because all the instruments require different entry angles to hold and retract. The current SILS procedure has been shown to take longer than standard multi-incision surgeries, cause more pain, and increase cost. This device reduces the cost and time associated with current SILS procedures. This device reduces the size of the incision and accommodates a variety of instrument angles, offering the attendant reduction in pain and time to heal. This device also allows the specimen to be removed through the incision, something that is not possible through a smaller port site.

As can be seen, there is a need for an improved device and method that reduces the cost and time associated with current SILS procedures and reduces the size of the incision and accommodates a variety of instrument angles, offering the attendant reduction in pain and time to heal.

### SUMMARY OF THE INVENTION

In one aspect of the present invention, a device to accommodate multiple ports through a single incision for laparoscopic surgery comprises an incision access member having a solid cylindrical body with body cavity wall engagement members disposed at opposed ends of the solid cylindrical body for accommodating surgical instruments inside a person's body when the incision access member is inserted through an incision in a body cavity wall of a person.

In another aspect of the present invention, a device to accommodate multiple ports through a single incision for laparoscopic surgery comprises an incision access member having a solid cylindrical body with body cavity wall engagement members disposed at opposed ends of the solid cylindrical body for accommodating surgical instruments inside a person's body when the incision access member is inserted through an incision in a body cavity wall of a person; the incision access member including bores extending lengthwise of and through the incision access member and through the opposed ends for receiving the surgical instruments; the body cavity wall engagement members being first and second annular flanges disposed at the opposed ends of the cylindrical body; the incision access member adapted to be removably inserted inside the single incision with a longitudinal axis of the incision access member disposed perpendicular to a plane of a body cavity wall of the person; the incision access member having a beveled recess in one of the ends to aid in the surgical procedure; the first annular flange configured to be removably received through the incision of the person in contactable relationship with an inner side of the body cavity wall exposed by the incision; and the second annular flange configured to be in contactable relationship with an outer side of the body cavity wall at the incision; the bores having different sized diameters to accommodate the reception of surgical instruments.

In a further aspect of the present invention, a method of accommodating multiple ports through a single incision for laparoscopic surgery include providing an incision access member having a solid cylindrical body, annular flanges and bores extending there through; removably inserting one of the annular flanges and the cylindrical body in the incision; securing the annular flanges to a body cavity wall at the incision; inserting multiple surgical instruments through the bores into body cavity of person; and inserting gas through the bores into body cavity of person.

These and other features, aspects and advantages of the present invention will become better understood with reference to the following drawings, description and claims.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of a new device to accommodate multiple ports through a single incision for laparoscopic surgery according to on implementation of the present invention.

FIG. 2 is a side elevation view of one aspect of the present invention with a cutaway.

FIG. 3 is a plan view of one aspect of the present invention.

FIG. 4 is an elevation view of one aspect of the present invention and a cross section of a body cavity and incision with the present invention being shown in use.

FIG. 5 is flowchart of the method of using multiple ports through a single incision for laparoscopic surgery with the device of FIG. 1.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

Broadly, embodiments of the present invention generally provide an incision access member that may include a solid cylindrical body operative to accommodate various surgical instruments inside a person's body when the incision access member is inserted through an incision in a body cavity wall of a person. Surgical tools may be inserted through bores of the solid cylindrical body to perform the single incision laparoscopic surgery (SILS) procedure.

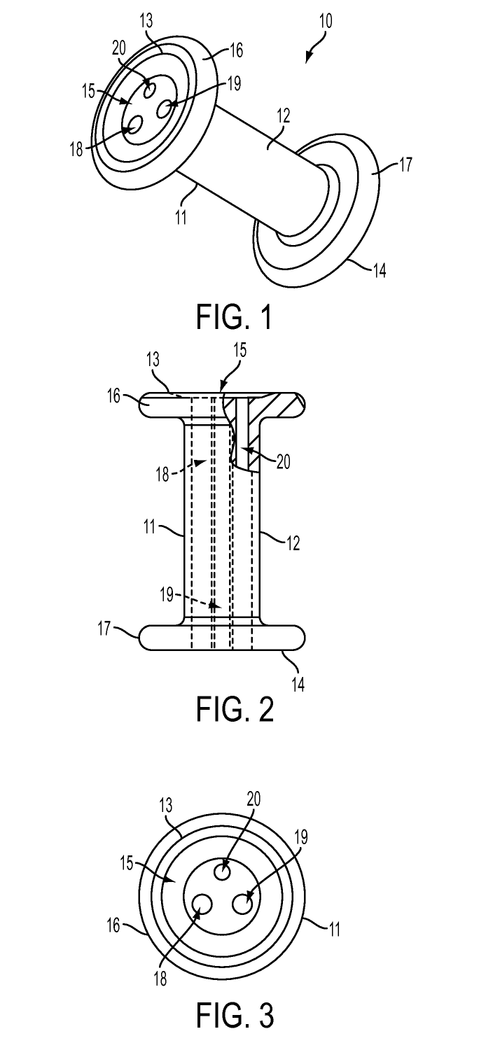

With reference now to the drawings, and in particular to FIGS. 1 through 5 thereof, a new device to accommodate multiple ports through a single incision for laparoscopic surgery embodying the principles and concepts of the present invention and generally designated by the reference numeral 10 will be described.

As best illustrated in FIGS. 1 through 3, the device 10 to accommodate multiple ports through a single incision for laparoscopic surgery generally comprises an incision access member 11 made of rigid or foam material and having a solid cylindrical body 12 with opposed ends 13, 14 for accommodating surgical instruments 26, 27 to operate inside a person's body when the incision access member 11 is inserted through a single incision 21 in a body cavity wall 23 of the person's body. In one implementation, incision access member 11 is constructed from an expandable thermal foam material, but in another implementation member 11 may also be constructed from polyvinyl chloride (PVC) or Acrylonitrile butadiene styrene (ABS) plastic.

As illustrated in FIGS. 1 and 2, the incision access member 11 may also include body cavity wall engagement members 16, 17, also referred to herein as “first annular flange 16 and second annular flange 17”, being conventionally attached to and integrally disposed at the opposed ends 13, 14 of the cylindrical body 12 and further includes bores 18-20 extending lengthwise of and through the incision access member 11 for receiving the surgical instruments 26, 27. In addition, the incision access member 11 may have a length of approximately 40 to 60 millimeters and may be removably disposed inside the single incision 21. A cavity of Body 12 may have a longitudinal axis which is generally perpendicular to a plane of the body cavity wall 23 when the incision access member 11 is inserted through the single incision 21 in a body cavity wall 23 of the person's body. When inserted, access member 11 may be compressed when inserted, and expand to seal the body cavity walls 23.

FIGS. 1 and 3 may depict the incision access member 11 having a beveled recess 15 in one of the ends 13, 14. The recess 15 aids in the surgical procedure by aiding in the seating and guiding of the surgical instruments 26, 27 into the bores 18-20 of the incision access member 11.

The body cavity wall engagement members 16, 17 may be conventionally or integrally attached to and disposed at the opposed ends 13, 14 of the cylindrical body 12. The first and second annular flanges 16, 17 may have a diameter of approximately 23 to 43 millimeters and the solid cylindrical body 12 may have a diameter of approximately 10 to 20 millimeters. The first and second flanges 16, 17 may extend outwardly generally perpendicular to the longitudinal axis of the solid cylindrical body 12.

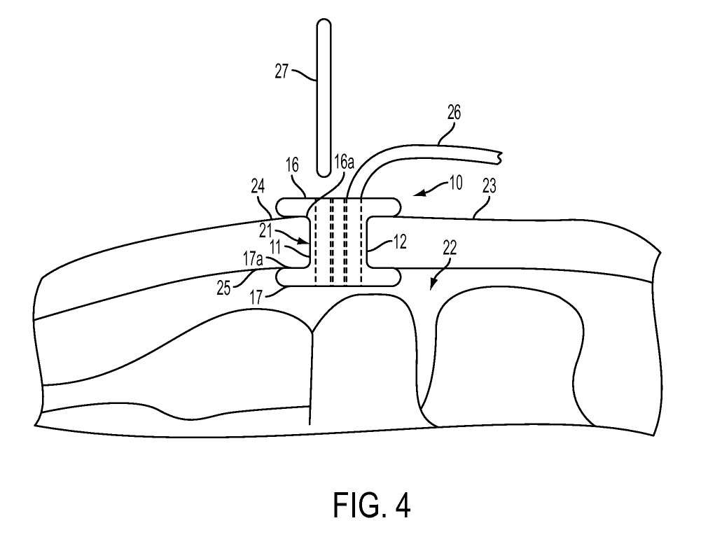

As illustrated in FIG. 4, the first annular flange 16 may be configured to be removably inserted through the single incision 21 of the person and has an inner side 16a facing the second annular flange 17 with the inner side 16a of the first annular flange 16 configured to be in contactable relationship with an inner side 24 of the body cavity wall 23 exposed by the single incision 21. The second annular flange 17 may have an inner side facing the first annular flange 16 with the inner side 17a of the second annular flange 17 configured to be in contactable relationship with an outer side 25 of the body cavity wall 23 at the single incision 21 to substantially secure the incision access member 11 with clamps (not shown) to the body cavity wall 23. The bores 18-20 may each have a different sized diameter ranging from 2 millimeters to 6 millimeters to accommodate the surgical instruments 26, 27 inserted through the incision member 11 such as a Carbon Dioxide gas tube 26.

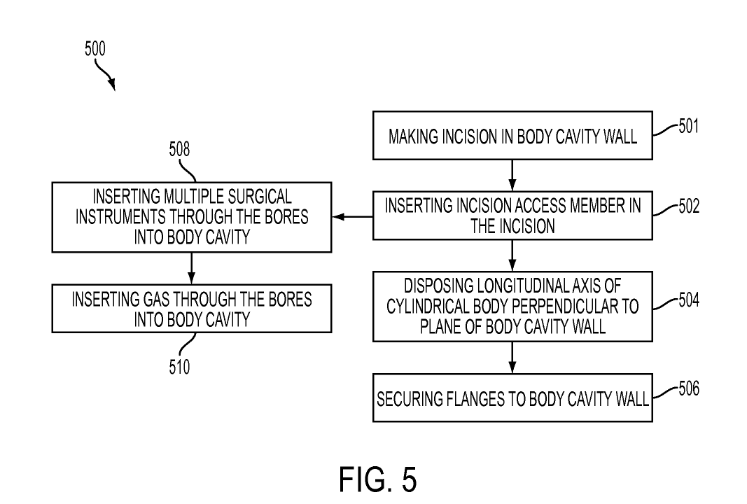

According to an exemplary embodiment as illustrated in FIG. 5, one implementation of the invention that includes a single incision laparoscopic surgery (SILS) procedure may be performed in the following manner described in process 500. A single incision 21 may be made at the bellybutton (block 501). The incision access member 11 may be inserted into the single incision 21 (block 502) with the longitudinal axis of the cylindrical body 12 being perpendicular to the plane of the body cavity wall (block 504). The incision access member 11 may be held in place by a clamp (not shown) to provide access inside the body cavity 22 of the person's body and an airtight seal (block 506). Standard surgical instruments 26, 27 may be inserted into the bores 18-20 (block 508), and a CO₂ gas connecting tube 26 may be connected at an end of one of the bores 18-20, preferably at the smallest one of the bores 18-20. The abdomen or any other body cavity 22 may then be insufflated with CO₂ gas by pumping Carbon Dioxide gas through one of the bores 18-20 (block 510). In one implementation of the single incision laparoscopic surgery (SILS) procedure, all of the instruments (not shown) to the procedure enter through the same point.

The insertion of the instruments 26, 27 may be aided as explained previously by the beveled recess 15 in one of the ends 13, 14 of the incision access member 11. The number and size of the bores 18-20 may be varied so that surgical instruments 26, 27 of various sizes can be extended through the bores 18-20 to perform various surgical procedures. The body cavity 22 may be insufflated through a bore 18-20. This device 10 may be used in any surgeries that would benefit from the availability of multiple ports through a single incision 21, including abdomen, chest and rectal surgeries.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

It should be understood, of course, that the foregoing relates to exemplary embodiments of the invention and that modifications may be made without departing from the spirit and scope of the invention as set forth in the following claims.

What is claimed is:

1. A device to accommodate multiple ports through a single incision for laparoscopic surgery comprising:
an incision access member having a solid cylindrical body with body cavity wall engagement members disposed at opposed ends of the solid cylindrical body, the incision access member operative to accommodate surgical instruments inside a person's body when the incision access member is inserted through an incision in a body cavity wall of the person's body.

2. The device to accommodate multiple ports through the single incision for laparoscopic surgery of claim 1, wherein the incision access member includes bores extending lengthwise through the incision access member and through the opposed ends to receive the surgical instruments.

3. The device to accommodate multiple ports through the single incision for laparoscopic surgery of claim 2, wherein the body cavity wall engagement members include a first and a second annular flanges disposed at the opposed ends of the cylindrical body.

4. The device to accommodate multiple ports through the single incision for laparoscopic surgery of claim 3, wherein the incision access member is adapted to be removably inserted inside the single incision with a longitudinal axis of the incision access member disposed perpendicular to a plane of the body cavity wall surface of the person's body, the incision access member having a beveled recess in one of the ends to aid in a surgical procedure.

5. The device to accommodate multiple ports through the single incision for laparoscopic surgery of claim 3, wherein the first annular flange is configured to be removably received through the incision of the person in contactable relationship with an inner side of the body cavity wall exposed by the incision, and the second annular flange is configured to be in contactable relationship with an outer side of the body cavity wall at the incision to substantially secure the incision access member to the body cavity wall.

6. The device to accommodate multiple ports through the single incision for laparoscopic surgery of claim 2, wherein the bores each have a different sized diameter to accommodate the reception of different sized surgical instruments.

7. The device to accommodate multiple ports through the single incision for laparoscopic surgery of claim 6, wherein one of the bores is adapted to receive a Carbon Dioxide gas tube at an end of the bore.

8. A device to accommodate multiple ports through a single incision for laparoscopic surgery comprising:
an incision access member having a solid cylindrical body with body cavity wall engagement members disposed at opposed ends of the solid cylindrical body for accommodating surgical instruments inserted inside a person's body via the incision access member when the incision access member is inserted through a single incision in a body cavity wall of a person, the incision access member including bores extending lengthwise through the incision access member and through the opposed ends to receive the surgical instruments, the body cavity wall engagement members having first and second annular flanges disposed at the opposed ends of the cylindrical body, the incision access member adapted to be removably inserted inside the single incision with a longitudinal axis of the incision access member disposed perpendicular to a plane of the body cavity wall of the person, the incision access member having a beveled recess in one of the opposed ends to aid in a surgical procedure, the first annular flange configured to be removably received through the single incision of the person in contactable relationship with an inner side of the body cavity wall exposed by the single incision, and the second annular flange configured to be in contactable relationship with an outer side of the body cavity wall at the single incision, the bores each having a different sized diameter to accommodate the reception of surgical instruments.

9. A method of accommodating multiple ports through a single incision for laparoscopic surgery comprising:
providing an incision access member having a solid cylindrical body, annular flanges and bores extending through the solid cylindrical body of the incision access member;
removably inserting one of the annular flanges and the cylindrical body of the incision access member in a single incision; and
securing the annular flanges to a body cavity wall at the single incision.

10. The method of accommodating multiple ports through the single incision for laparoscopic surgery of claim 9, wherein the removably inserting the incision access member further includes inserting the incision access member with a longitudinal axis of the incision member being perpendicular to a plane of the body cavity wall.

11. The method of accommodating multiple ports through the single incision for laparoscopic surgery of claim 9, wherein the securing the annular flanges further includes engaging an inner side of one of the annular flanges to an inner side of the body cavity wall at the single incision and engaging an inner side of the other of the annular flanges to the outer side of the body cavity wall at the single incision.

12. The method of accommodating multiple ports through the single incision for laparoscopic surgery of claim 9 comprising inserting multiple surgical instruments through the bores into a body cavity of a person.

13. The method of accommodating multiple ports through the single incision for laparoscopic surgery of claim 12 comprising inserting gas through the bores into the body cavity of the person.
